(19) United States
(12) Patent Application Publication
(10) Pub. No.: US 2010/0228280 A1
(43) Pub. Date: Sep. 9, 2010

(54) METHODS AND DEVICES FOR TREATMENT OF LUMENAL SYSTEMS

(76) Inventors: Adam Groothuis, Swampscott, MA (US); Edward I. McNamara, Chelmsford, MA (US); Peter Markham, Kingston, NH (US); Elazer R. Edelman, Brookline, MA (US)

Correspondence Address: EDWARDS ANGELL PALMER & DODGE LLP, P.O. BOX 55874, BOSTON, MA, 02205, US

(21) Appl. No.: 12/400,041

(22) Filed: Mar. 9, 2009

(51) Int. Cl. A61M 29/02; A61M 25/10; A61F 2/01

(52) U.S. Cl. 606/200; 604/96.01; 604/103.1

(57) ABSTRACT

The invention provides a variety of catheters and methods for using such catheters. An exemplary catheter includes an elongate body having a proximal end a distal end. The elongate body defines a longitudinal axis of the catheter. The exemplary catheter further includes at least one inflatable member disposed on the elongate body proximate the distal end of the elongate body. The interior of the inflatable member is in fluid communication with an inflation lumen in the elongate body. The exemplary catheter also includes a first filter adapted and configured to capture emboli in a patient's bloodstream as the bloodstream passes through the first filter along a first direction. The exemplary catheter further includes a second filter adapted and configured to capture emboli in a patient's bloodstream as the bloodstream passes through the second filter along a second direction, wherein the second direction is generally opposite to the first direction.

### BACKGROUND OF THE INVENTION

1. Field of the Invention

The present invention relates to a method and system for treating the luminal system of a patient. Particularly, the present invention is directed to a method and system for treating aortic stenosis and potential capture of debris.

2. Description of Related Art

Aortic stenosis is caused by the hardening of the aortic valve leaflets. Hardening of the aortic valve leaflets results in increased flow resistance, and thus, the force that must be exerted by the left ventricle to eject blood to the rest of the body. The hardening of the leaflets is caused by artherogenic agents that are absorbed, as well as the presence of chronic inflammation.

A variety of methods and systems are known in the art for treating aortic stenosis. Of such devices, many are directed to open surgical techniques as well as complex percutaneous techniques that are difficult to perform. Notably, patients with severe aortic stenosis left untreated have a life expectancy of less than five years.

Open surgical techniques used to correct for aortic stenosis typically include open-heart surgery. Aortic valve replacement is the primary treatment for severe aortic stenosis. Valves from animals, (e.g., pigs), may be used in such procedures to replace an aortic valve in a human. In order for an aortic replacement valve to be implanted, a surgeon surgically replaces the aortic valve with such as substitute valve. This requires open-heart surgery which involves opening a patient's sternum and placing the patient on a heart bypass machine while the valve is replaced.

A second procedure that has been used to reduce aortic stenosis involves percutaneous aortic valve replacement using a stent valve. A stent valve is typically delivered through a large bore access site and is placed at the native valve pinning the leaflets. By replacing the valve in this manner, the gradient through the valve may be substantially reduced. Although the percutaneous placement of a stent valve is generally successful in reducing the valve gradient, this technique has significant drawbacks. Specifically, patients that undergo this procedure experience procedural success about sixty percent of the time. Moreover, during placement of the stent valve, it is possible that debris are released, which greatly increases the risk of an embolism. This causes strokes in approximately ten percent of patients that undergo percutaneous aortic valve replacement. Furthermore, any time open chest procedures are performed, there are associated risks and potential lengthy hospitalization.

A further procedure that is temporarily successful in correcting for aortic stenosis is aortic valvuloplasty. During an aortic valvuloplasty procedure, a valvuloplasty balloon is inserted across the valve and inflated to break up hardened deposits in the leaflets, and cause the valve leaflets to become more flexible. However, as with the stent valve procedure mentioned above, there is significant risk of embolism and stroke. While percutaneous devices exist to capture emboli in general, the geometry of a patient's coronary artery system proximate the aortic valve is very complex and subject to constant reversals in blood flow as a result of operation of the heart.

Thus, there still remains a continued need in the art for effective and safer minimally invasive techniques for treating aortic stenosis. The present invention solves these problems, as described herein.

### SUMMARY OF THE INVENTION

Advantages of the present invention will be set forth in and become apparent from the description that follows. Additional advantages of the invention will be realized and attained by the methods and systems particularly pointed out in the written description and claims hereof, as well as from the appended drawings.

To achieve these and other advantages and in accordance with the purpose of the invention, as embodied herein, the invention includes a first embodiment of a catheter. The catheter includes an elongate body having a proximal end a distal end. The elongate body defines a longitudinal axis of the catheter. The catheter further includes at least one inflatable member disposed on the elongate body proximate the distal end of the elongate body. The interior of the inflatable member is in fluid communication with an inflation lumen in the elongate body. The catheter also includes a first filter disposed on the elongate body at a location proximal to the inflatable member. The first filter is adapted and configured to capture emboli in a patient's bloodstream as the bloodstream passes through the first filter along a first direction. The catheter further includes a second filter disposed on the elongate body at a location proximal to the inflatable member, but distal to the first filter. The second filter is adapted and configured to capture emboli in a patient's bloodstream as the bloodstream passes through the second filter along a second direction, wherein the second direction is generally opposite to the first direction.

In accordance with a further aspect of the aforementioned embodiment, the second filter can be adapted and configured to expand and contract along a direction generally transverse to the longitudinal axis of the catheter. In accordance with a preferred embodiment, the second filter is adapted and configured to expand and contract in response to a change in direction of a patient's blood flow and/or a change in a patient's local blood pressure. In accordance with another embodiment, the second filter may be adapted and configured to adjust in size in response to local pressure gradients in a patient's bloodstream. In accordance with a preferred embodiment, the second filter is adapted and configured to selectively expand and contract.

In accordance with still a further aspect, the second filter can be at least partially disposed within the first filter. If desired, the first filter and second filter can be adjusted in size, such as by adjusting a transverse dimension or diameter of the filters. In accordance with one embodiment, the first filter and/or second filter may include radiopaque material to facilitate visualization of the filters during a procedure in which the filters are deployed.

In accordance with still a further embodiment, the first filter and second filter can be displaced along the longitudinal axis with respect to the at least one inflatable member. For example, the first filter and second filter can be attached to a tubular member that is adapted and configured to receive the elongate body through a lumen defined by the tubular member. The tubular member can thus be translated longitudinally with respect to the elongate body as desired.

In accordance with yet another embodiment, the at least one inflatable member can include an undulating exterior surface defining at least one longitudinal channel therein. Preferably, the channel is sufficient to permit perfusion in a patient's blood vessel when the at least one inflatable member is expanded. In accordance with a particular embodiment, the catheter may include a plurality of inflatable members that cooperate to define at least one perfusion channel on the exterior of the catheter when the inflatable members are inflated. Similarly, the plurality of inflatable members may be adapted and configured to close and open the perfusion channel. By way of further example, the catheter may include a plurality of inflatable members that can be selectively expanded serially or in parallel. If desired, the one or more inflatable members may include polymeric material such as nylon.

In accordance with still another embodiment, the distance between the at least one inflatable member and the second filter is substantially the same as the distance between a patient's aortic valve and the entrance to the patient's coronary sinus. In accordance with another embodiment, the distance between the at least one inflatable member and the second filter is any desired distance, or may be adjusted. In accordance with another embodiment, a catheter is provided adapted and configures for use in neuro-thrombectomy procedures, and/or in stroke cases generally.

In accordance with yet another embodiment, the catheter further includes means for ejecting pressurized liquid proximate the distal end of the catheter. Preferably, the pressurized liquid exits the device in the form of one or more jets sufficient to remove debris from the walls of a luminal system of a patient. If desired, the means for ejecting liquid may include a plurality of openings in the exterior surface of the catheter in fluid communication with a source of pressurized fluid. In accordance with one embodiment, the means for ejecting liquid is adapted and configured to eject liquid in a direction generally transverse to the longitudinal axis. However, additional embodiments may be adapted and configured to eject liquid at various angles with respect to the longitudinal axis. The means for ejecting liquid may, for example, include a plurality of openings on the surface of the inflatable member. For example, the openings on the surface of the inflatable member may be in fluid communication with a source of pressurized fluid that is not in fluid communication with fluid used to inflate the at least one inflatable member.

In further accordance with the invention, a catheter is provided. The catheter includes an elongate body having a proximal end a distal end. The elongate body defines a longitudinal axis of the catheter. The catheter further includes a first filter disposed on the elongate body at a location proximal to the distal end, wherein the first filter is adapted and configured to capture emboli in a patient's bloodstream as the bloodstream passes through the first filter along a first direction. The catheter further includes a second filter disposed on the elongate body at a location proximal to the distal end and distal to the first filter. The second filter is adapted and configured to capture emboli in a patient's bloodstream as the bloodstream passes through the second filter along a second direction, wherein the second direction is generally opposite to the first direction.

In accordance with a further aspect, the catheter may optionally includes at least one inflatable member disposed on the elongate body proximate the distal end of the elongate body. The interior of the inflatable member is preferably in fluid communication with an inflation lumen in the elongate body.

In further accordance with the invention, a method is provided for treating a patient's luminal system. The method includes providing an embodiment of a catheter as described herein, disposing the distal end of the catheter in a patient's luminal system, and treating the luminal system of the patient using the catheter, wherein the filters are used to collect debris resulting from the procedure.

In accordance with a further aspect, if the catheter is provided with at least one inflatable member, the method may further include expanding the at least one inflatable member to assist in the treatment procedure.

In accordance with a further aspect, the distal end of the catheter may be disposed proximate a valve in the patient's luminal system. For example, the valve can be the patient's aortic valve. In accordance with this embodiment, the first filter of the device can be disposed at a location downstream from the patient's aortic root to capture emboli. Accordingly, the second filter can be expanded to prevent the emboli from being directed into the patient's coronary sinus arteries. The second filter may be expanded and contracted in response to a change in direction of a patient's blood flow, a patient's blood pressure, local pressure gradients in a patient's bloodstream and/or the second filter may be selectively and controllably expanded and contracted. If desired, the second filter can be disposed at least partially within the first filter. By way of further example, the first and second filters may be translated longitudinally with respect to the inflatable member, as well as each other.

In accordance with still a further aspect, the method may include defining a perfusion channel proximate the exterior of the inflatable member. By way of further example, the method may also include ejecting pressurized liquid proximate the distal end of the catheter to remove debris from a target region, such as a valve of a patient's luminal system. The ejecting step preferably includes directing pressurized liquid through a plurality of openings disposed on the surface of the inflatable member.

It is to be understood that the foregoing general description and the following detailed description are exemplary and are intended to provide further explanation of the invention claimed.

The accompanying drawings, which are incorporated in and constitute part of this specification, are included to illustrate and provide a further understanding of the method and system of the invention. Together with the description, the drawings serve to explain principles of the invention.

### BRIEF DESCRIPTION OF THE DRAWINGS

FIGS. 1(A)-1(B) are schematic plan and end views of a first representative embodiment of a catheter in accordance with the present invention.

FIG. 2 is a partial schematic view of a distal portion of a second representative embodiment of a catheter in accordance with the present invention.

FIG. 3 is a schematic view of a portion of the catheter of FIG. 1 further illustrating exemplary filtering mechanisms.

FIG. 4 is a schematic view of an embodiment of a catheter in accordance with the present invention having a plurality of filters that can be translated toward or away from each other.

FIG. 5 is a schematic view of a portion of a representative catheter made in accordance with the present invention including a deliverable prosthesis.

FIGS. 6-9 are illustrations of an exemplary method carried out in accordance with an embodiment of the invention.

FIG. 10 is an illustration of a portion of another representative embodiment of a device and associated method in accordance with the invention.

### DETAILED DESCRIPTION OF PREFERRED EMBODIMENTS

Reference will now be made in detail to the present preferred embodiments of the invention, examples of which are illustrated in the accompanying drawings. The method and corresponding steps of the invention will be described in conjunction with the detailed description of the system.

The present invention provides methods and systems that alleviate the above-referenced shortcomings in the art. The devices and methods presented herein may be used for treating the luminal system of a patient. The present invention is particularly suited for treatment of valves in the luminal system of a patient, such as the aortic valve.

In accordance with the invention, a first embodiment of a catheter is provided including an elongate body having a proximal end a distal end, at least one inflatable member disposed on the elongate body, and first and second filters disposed on the elongate body.

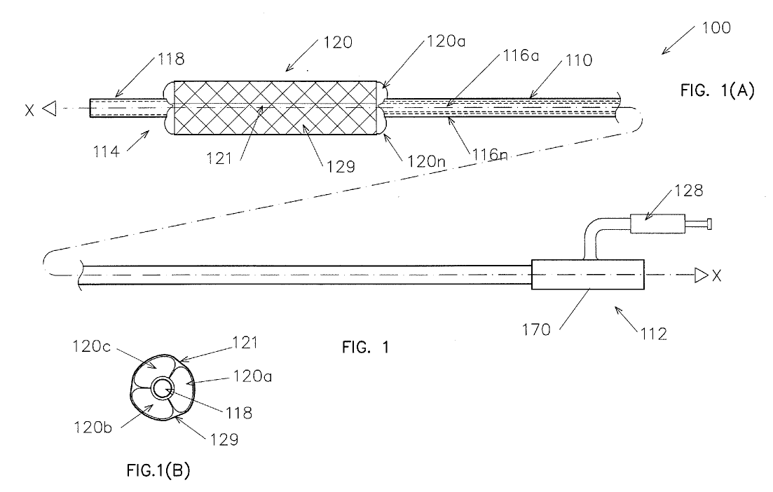

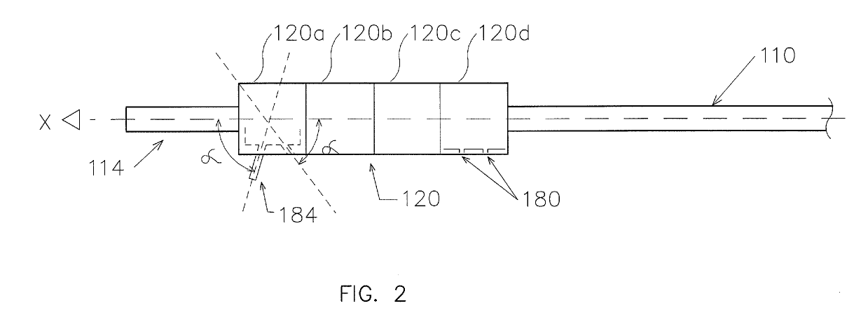

For purpose of explanation and illustration, and not limitation, a partial view of an exemplary embodiment of a catheter in accordance with the invention is shown in FIG. 1 and is designated generally by reference character 100. Other embodiments of a catheter in accordance with the invention, or aspects thereof, are provided in FIGS. 2-10, as will be described.

As depicted in FIG. 1, catheter 100 includes an elongate body 110 having a proximal end 112 and a distal end 114. The elongate body 110 defines a longitudinal axis X of the catheter 100. The catheter 100 further includes at least one inflatable member 120 disposed on the elongate body 110 proximate the distal end 114 of the elongate body 100. Elongate body 110 may also include one or more lumens 116a-n along the length thereof for directing fluids for inflating one or more inflatable members 120a-120n, or for acting as a conduit for a pull or push wire, among other purposes.

Elongate body 110 may be made in a variety of ways and from a variety of materials. For example, elongate body 110 may be made from a variety of materials, including metal, plastic and composite materials. Metal tubes such as stainless steel hypotubes can be used for one or more portions of elongate body 110 for enhanced pushability alone or in combination with other suitable materials. If metal tubular components are used to make elongate body 110 they are preferably coated with a lubricious material such as PTFE, other hydrophobic materials or hydrophilic materials. Multilayered polymeric tubes can also be used to form elongate member 110 that can be formed by coextrusion, dipping processes, or by shrinking tubing layers over one another over a mandrel. Moreover, polymeric tubular members can also be formed by charging a mandrel with static electricity, applying plastic in powder or granular form to the mandrel to form a layer of plastic over the mandrel, and by heating the mandrel to cause the particles to fuse. Multilayered polymeric tubes can also be used that include metallic or nonmetallic braiding within or between layers of the tube. A carbon tube can also be used, as well as fiber-reinforced resin materials. In accordance with another embodiment, elongate body 110 may be provided with a decreasing stiffness along its length from proximal end 112 to distal end 114. As will be further appreciated by those of skill in the art, elongate body 110 may also include a multiple-lumen extrusion including two, three, four, or more lumens along part of or substantially the entire length of elongate body 110. Moreover, stiffening members such as stiffening wires can be used at various locations along elongate body to provide stiffness transitions between relatively stiffer regions and less stiff regions, as well as proximate regions of stress concentration, such as guidewire exit ports and the like. In accordance with one embodiment, a guidewire lumen 118 is provided along substantially the entire length of elongate body 110 as with typical over the wire (“OTW”) catheters. In accordance with another embodiment, a guidewire lumen 118 is provided only proximate the distal region of elongate body 110 to permit use of catheter 100 as a rapid exchange “RX”) catheter.

As further depicted in FIG. 1, the catheter 100 further includes one or more inflatable members 120a-n. In accordance with a preferred embodiment, the catheter includes three inflatable members 120a-c. Each of the inflatable member 120a-c is in fluid communication with an inflation lumen 116a-c. As depicted, inflatable members 120a-c are generally elongate and may be selectively inflated with a working fluid. When inflated, adjacent inflatable members 120 can define a channel 121 therebetween, which may be used to permit perfusion between the catheter 100 and wall of the luminal passage of the patient.

It will be further appreciated that inflatable member 120 can similarly comprise a single balloon having a plurality of lobes similarly defining perfusion channels between the lobes. By way of further example, if desired, the interior of elongate body 110 can define a perfusion channel therethrough that may include passages through the wall of the elongate body to permit perfusion from a region distal to the catheter to a region proximal to the inflatable members 120. If desired, inflatable members 120a-n may be selectively expanded serially or in parallel. For example, each inflatable member 120a-n may be selectively actuable such that they may be inflated sequentially or simultaneously. By way of further example, while inflatable members 120a-n are depicted as being generally elongate and parallel to one another, they may alternatively be arranged so as to be longitudinally arranged as depicted in FIG. 2. In accordance with that embodiment, the inflatable members 120a-n are preferably generally toroidally-shaped.

Inflatable members 120a-n can be made from a variety of materials. For purpose of illustration and not limitation, inflatable members 120 can be made from a poly ether block amide (“PEBA”), nylon, Hytrel, PU, PEEK, PE or a variety of other materials. Inflatable member 120 can be attached to distal end 114 of elongate body 110 by way of adhesive bond (such as by way of adhesive that it polymerized by exposure to light (e.g, ultraviolet light)), fusion bond, or preferably by welding. Thus, if inflatable member 120 is made of nylon, it is advantageous for the outer surface 115 of elongate body 110 to be made of a material compatible for a welded bond therebetween.

By way of further example, an inflation device 128 may be provided for inflating the inflatable member 120. The inflation device 128 can be, for example, a syringe or a flexible reservoir that is connected to a proximal end 112 of elongate body 110 and actuated to inflate inflatable member 120.

As further depicted in FIGS. 1(A)-1(B), if desired, the inflatable member(s) can be provided with a mesh covering 129. Such a covering is advantageous because it helps define a perfusion channel between itself and the balloons by acting to hold back the vessel wall or valve leaflets of the patient. Mesh 129 can be made from any suitable material, including but not limited to polymeric and composite materials. In accordance with one embodiment, mesh 129 is made from a flexible material that can expand and contract with the inflation and deflation of inflatable members 120a-n.

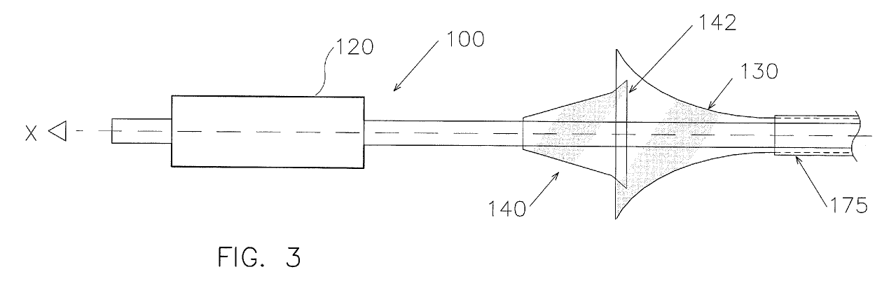

As depicted in FIG. 3, catheter 100 also includes a first filter 130 disposed on the elongate body 110 at a location proximal to the inflatable member(s) 120. The first filter 130 is adapted and configured to capture emboli or other debris in a patient's bloodstream as the bloodstream passes through the first filter 130 along a first direction. The catheter further includes a second filter 140 disposed on the elongate body 110 at a location proximal to the inflatable member(s), but distal to the first filter 130. The second filter 140 is adapted and configured to capture emboli or other debris in a patient's bloodstream as the bloodstream passes through the second filter 140 along a second direction, wherein the second direction is generally opposite to the first direction. In other words, first filter 130 and second filter 140 are configured to capture and preferably isolate debris in a patient's bloodstream. This filter arrangement is particularly advantageous when used in a region of blood flow that undergoes reversal in direction, such as proximate the aortic valve.

As will be appreciated by those of skill in the art, when delivering the filters 130, 140 to a target location within the luminal system of a patient, they are preferably in a collapsed form, and then selectively deployed. In accordance with a preferred embodiment and as depicted in FIG. 3, the second filter 140 can be at least partially disposed within the first filter 130. In accordance with one embodiment, the first filter 130 and/or second filter 140 preferably include radiopaque material to facilitate visualization of the filters during a procedure in which the filters are deployed.

Preferably, both filters 130, 140 are adapted and configured to expand and contract along a generally radial direction generally transverse to the longitudinal axis X of the catheter 100 in a manner similar to an umbrella. Each of filters 130, 140 can be selectively deployed in a variety of manners. In accordance with one embodiment, one or both of the filters can be actuated with a pushwire or other actuator, wherein each filter is operably coupled with a push wire and/or a pull wire that may be disposed within a pushwire lumen, for example, within elongate body 110. In accordance with a preferred embodiment, one or both filters may be adapted and configured to open and close in response to local flow conditions. For example, in accordance with a particularly preferred embodiment, second filter 140, which can be adapted and configured to close inside of filter 130 to capture debris dislodged from first filter 130 during conditions of flow reversal, can be adapted and configured to be pushed open by the reversing flow, causing it to open like an umbrella. When the flow reverses yet again, blood can urge the second filter 140 closed, such that debris flow into first filter 130. The geometry and structure of second filter 140 can be optimized to facilitate this operation. For example, as depicted in FIG. 3, second filter 140 can be provided with a flared periphery 142 that, when closed, helps funnel blood back into the filter 140 during a flow reversal, causing it to open. Similarly, such a flared periphery 142 can help increase the force of blood flow in the opposite direction on the outside of the filter to help it close. In addition or in the alternative to a flared opening, one or both filters 130, 140 can be made from a resilient material that self-expands upon being released from a retractable sheath 175, described further below. Accordingly, the openability and closeability of filters 130, 140 can be tailored to the particular flow conditions in the patient's lumenal system, for example, by choosing a stiffer or less stiff material. Filters 130, 140 can also include shape memory material (such as various nickel-titanium alloys known in the art) that helps the filters to deploy when in the patient's blood stream.

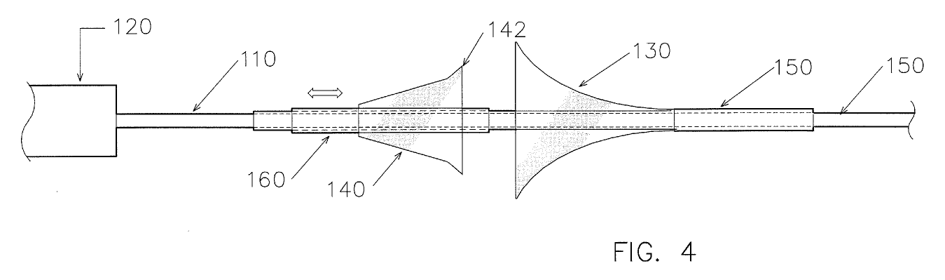

If desired, as depicted in FIG. 4, one or both of filters 130, 140 may be disposed on the same or adjacent or overlapping sleeves 150, 160 that are adapted and configured to be translated over elongate body 110. Permitting longitudinal between the filters 130, 140 and elongate body 110 can be advantageous, when the distance between the inflatable member(s) 120 and the filters 130, 140 need to be adjusted during the procedure. Sleeves 150, 160 may be made in a variety of manners as with elongate member 110 described above. Preferably, sleeves include a multi-layered co-extrusion, such as those described in U.S. Pat. No. 6,464,683 to Samuelson or U.S. Pat. No. 5,538,510 to Fontirroche. Each of the aforementioned patents is incorporated by reference herein in its entirety.

An actuator 170 may be used to produce relative movement between the filters 130, 140 and the elongate member 110 and/or between filters 130, 140. Actuator 170 can take on a variety of forms. For example, a relatively simple push-pull actuator may be provided (as depicted). Moreover, it is also possible to use other actuators as are known in the art, such as threaded rotating actuators as described in U.S. Pat. No. 6,488,694 to Lau and U.S. Pat. No. 5,906,619 to Olson, each of which is incorporated by reference herein in its entirety. In accordance with one embodiment, the distance between the inflatable member(s) 120 and the second filter 140 is substantially the same as the distance between a patient's aortic valve and the beginning of the patient's aortic root. Alternatively, the distance between the inflatable members and either filter can also be any other desired distance. Such a catheter 100 would be suitable for removal of debris proximate a patient's aortic valve, as described in further detail below. Regarding initial deployment, filters 130, 140 may be delivered on catheter 100 within one or more sheaths 175. The sheath may be withdrawn, for example, by retracting the sheath using a pull wire and actuator as described above.

In accordance with yet another embodiment, the catheter may include means for ejecting pressurized liquid proximate the distal end of the catheter. Preferably, the pressurized liquid exits the device in the form of one or more jets sufficient to remove debris from the walls of a luminal system of a patient.

For purposes of illustration and not limitation, as embodied herein and as depicted in FIG. 2, catheter 100 may be provided with one or more openings 180 defined therein, preferably proximate or integrated with inflatable member(s) 120. Openings 180 may be defined in the exterior surface of the catheter 100 in fluid communication with a source of pressurized fluid (not shown). In accordance with one embodiment, openings 180 are preferably adapted and configured to eject liquid (such as saline or other suitable liquid) in a direction generally transverse to the longitudinal axis X of catheter 100. However, additional embodiments may be adapted and configured to eject liquid at various angles with respect to the longitudinal axis X. The means for ejecting liquid may, for example, include a plurality of openings 180 on the surface of the inflatable member(s) 120. For example, the openings 180 on the surface of the inflatable member(s) 120 may be in fluid communication with a source of pressurized fluid that is used to inflate the inflatable members 120, or a different source of pressurized fluid, as desired. For example, openings 180 may simply be small openings in the inflatable member 120 that eject a jet of inflation fluid after a predetermined pressure in the inflatable member 120 has been achieved.

Openings 180 and a fluid source in communication therewith may be used to eject high speed jets 184 at debris lodged on vessel walls of a patient (at any suitable angle a with respect to axis X). The high speed jets 184 can be used to dislodge such debris, which are in turn carried by the patient's blood stream into filter 130 and/or 140, depending on the local flow conditions. In accordance with the embodiments herein, the ability to eject a fluid as described may be used to clean valve leaflets of debris, restoring their flexibility. For example, valve leaflets can be displaced from their normal location to a location toward the vessel wall by one or more inflatable members, and cleaned accordingly. Preferably, the leaflets are not pinned against the vessel wall to permit cleaning fluid to reach both sides of the leaflets by way of fluid jets. Similarly, it is believed that fluid ejected from the device can be directed through the leaflet tissue itself to the other side of the leaflet to further remove debris. Also, it is believed that the act of expanding the inflatable member against the leaflets causing them to flex will also help to break up hardened deposits.

As will be further appreciated by those of skill in the art, additional embodiments of catheters are provided that include certain features described above in combination with other features.

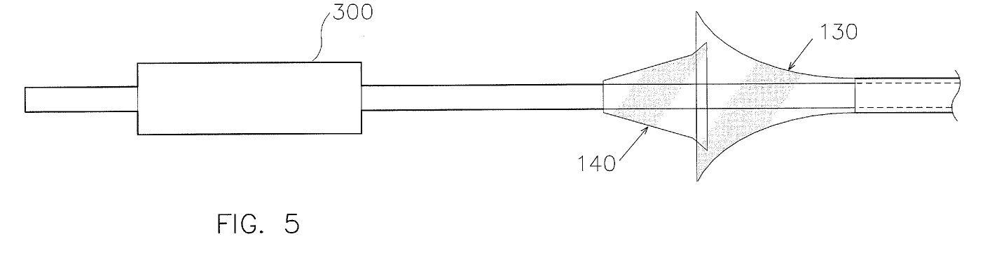

In accordance with a first example, a catheter may be provided sharing many of the features described above with respect to catheter 100. However one significant difference is that no inflatable member 120 is provided. Instead, filters 130, 140 (such as depicted in FIG. 3 or 4) may be used in a variety of applications such as treatment of acute stroke, the capture of freshly released thrombus, stroke, and cases of neuro thrombectomy, as well as treating other valves within the luminal system of a patient, among other applications. For example, a catheter may be provided adapted and configures for use in neuro-thrombectomy procedures, and/or in stroke cases generally. For such an application, the scale of the catheter 100 would be reduced (particularly in diameter and cross-sectional profile) and the filtering system could be used to capture thrombus. The filtering systems embodied herein may be used in combination with other procedures, such as stent valving and/or valvuloplasty. Moreover, catheter 100 can include a deliverable prosthesis 300 as depicted in FIG. 5 in place of or in addition to the inflatable members, depending on the preferred method of deployment.

Any surface of various components of the catheters described herein or portions thereof can be provided with one or more suitable lubricious coatings to facilitate procedures by reduction of frictional forces. Such coatings can include, for example, hydrophobic materials such as PolyTetraFluoroEthylene (“PTFE”) or silicone oil, or hydrophilic coatings such as Polyvinyl Pyrrolidone (“PVP”). Other coatings are also possible, including, echogenic materials, radiopaque materials and hydrogels, for example.

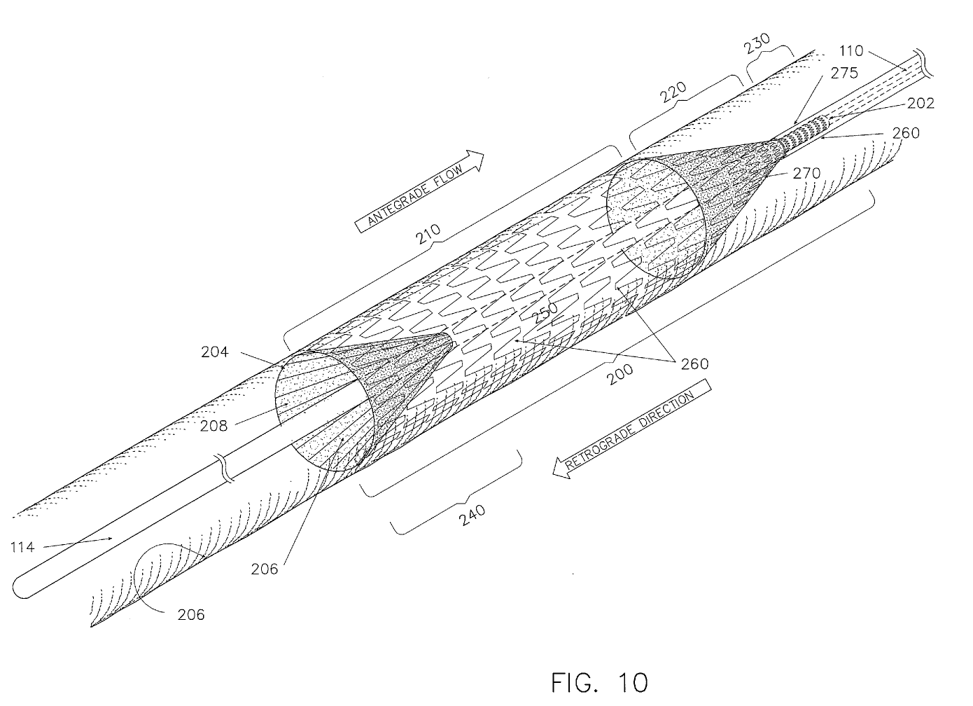

In further accordance with the invention, FIG. 10 illustrates a further embodiment of a filtering mechanism for capturing emboli. As illustrated, a catheter including an elongate body 110 is provided. However, in lieu of filters 130, 140, a generally cylindrical filter 200 is provided for capturing emboli. Filter 200 can be used in combination with inflatable members 120a-n for purposes of treating the aortic valve as described previously.

As depicted, filter 200 is a self-expanding structure. Filter 200 may include a plurality of expandable scaffolding rings 260 that self-expand against the vessel wall 205. The illustrative expandable scaffolding rings 260 are not intended to be limiting, but merely illustrative to demonstrate an exemplary structure that can be used to cause expansion of a generally cylindrically-shaped filter. Expandable scaffolding rings 260, if used, may be made from shape memory material (e.g., nickel-titanium alloys or other materials) such that the rings expand when a retractable sheath 275 is withdrawn along a proximal or distal direction, as desired. Filter 200 further includes a circumferential wall 270 that may be made from any desired material, that permits the passage of blood therethrough, but not emboli. This structural approach can also be used to make filters 130, 140.

In use, catheter 100 is introduced to a target location, such as proximate the aortic valve. Next, sheath 275 is withdrawn to cause the distal portion 210 filter 200, and the associated portion of wall 270, to expand against the vessel wall 205. Intermediate portion 220 of filter is only partially deployed, and proximal portion 230 of filter preferably remains within the distal portion of the sheath 275. At this point, blood is free to flow into the mouth 206 of filter, and out through the wall portion 270 of the filter in the filter's intermediate region 220. Mouth 206 of filter 200 preferably includes a conical valve 240 that tapers inwardly along the antegrade direction (as presently illustrated) having a plurality of leaflets 208 that urge against the elongate body 110. This design is particularly advantageous for the reversals in flow that accompany the arterial system immediately downstream from the aorta. When performing a procedure, debris removed from the aortic region will be carried into mouth 206 of filter 200 during antegrade flow. Shortly thereafter, when the flow direction reverses in a retrograde direction, the debris will be trapped within filter 200. When the procedure is complete, the sheath 275 may be urged back over the filter 200, causing it to collapse, and trapping the debris inside the filter 200 and sheath 275, thus permitting safe removal of the debris from the patient.

In further accordance with the invention, a method is provided for treating a patient's luminal system.

For purposes of illustration, and not limitation, the method includes providing an embodiment of a catheter (e.g., 100, as described herein), disposing the distal end of the catheter in a patient's luminal system, and treating the luminal system of the patient using the catheter, wherein the filters are used to collect debris resulting from the procedure. The following description illustrates use of catheter 100.

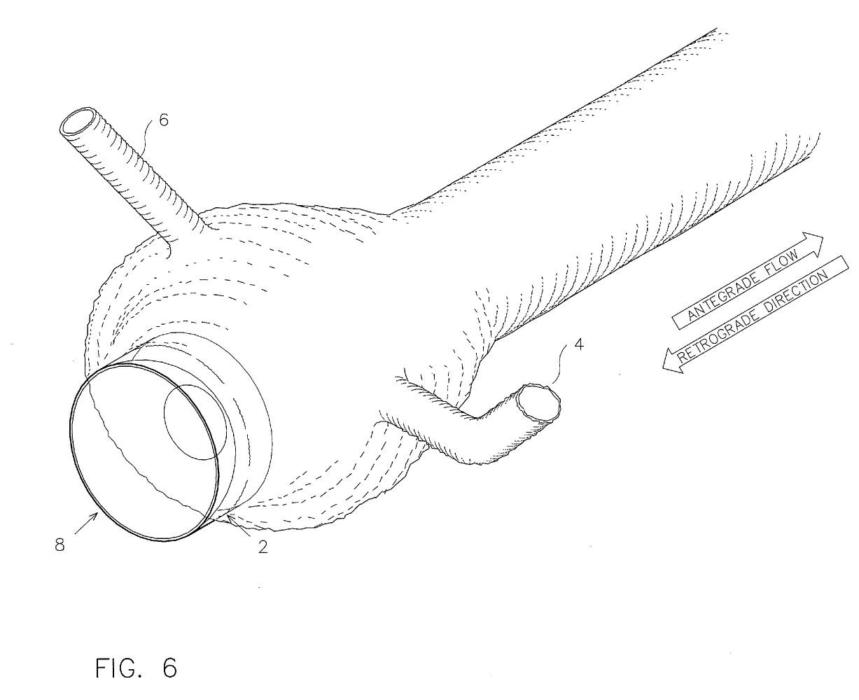

In accordance with this illustration of the method, catheter 100 will be used to perform a beneficial procedure on a patient's aortic valve 2 located proximate the heart 8. As depicted in FIG. 6, the patient's aortic valve 2 is presented, as well as the entrances to the patient's coronary sinus and cerebral arteries 4, 6. Applicant has observed that the region of the patient's bloodstream proximate the aortic valve (as depicted in FIG. 6) is subject to reversals in blood flow in accordance with the patient's heartbeat. For example, in between heartbeats blood flows in the retrograde direction back toward the patient's heart during diastole to fill the coronaries. Use of a catheter 100 can permit treatment of the aortic valve 2 in order to loosen debris, yet still capture such debris in this region of complex bloodflow and thus preventing such debris from entering into the patient's coronary sinus and cerebral arteries. It will be appreciated that filter 200 can be used in lieu of filters 130, 140 with respect to any embodiment disclosed herein.

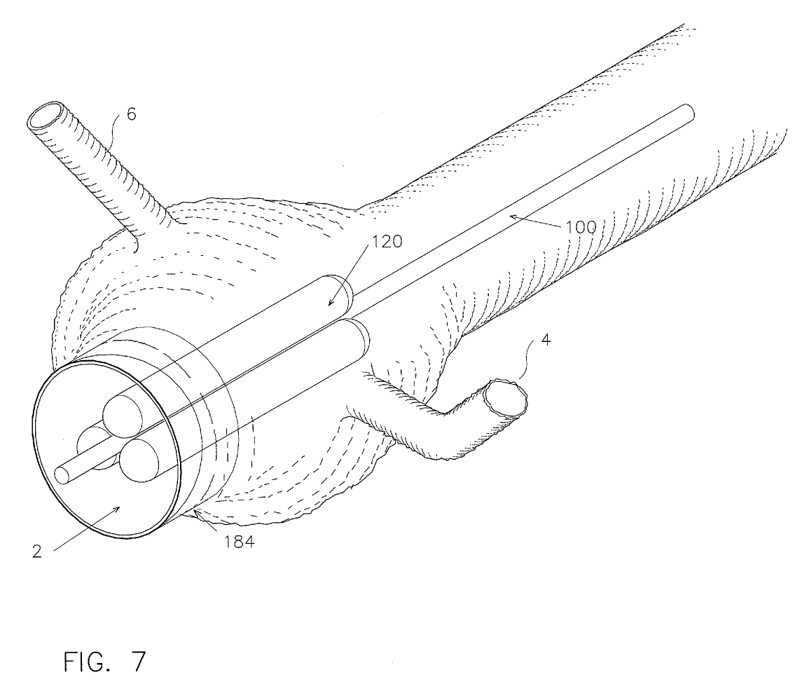

As depicted in FIG. 7, catheter 100 can be introduced into this region. Inflatable member(s) 120 can then be advanced into the aortic valve. Inflatable member(s) 120 can then be inflated, causing deposits formed on the valve leaflets to crack and loosen. Pressurized fluid jets 184 further act to loosen debris, and cause them to be swept up in the patient's bloodstream, where they are caught by filters 130, 140 (or 200) at a downstream location. While the momentum of the fluid jets 184 can physically pry foreign matter from the leaflets of the aortic valve, it is also believed that fluid may pass through the leaflet structure to further remove unwanted materials. Preferably, the leaflets of the aortic valve 2 are separated, but not pinned against the vessel wall, during the procedure.

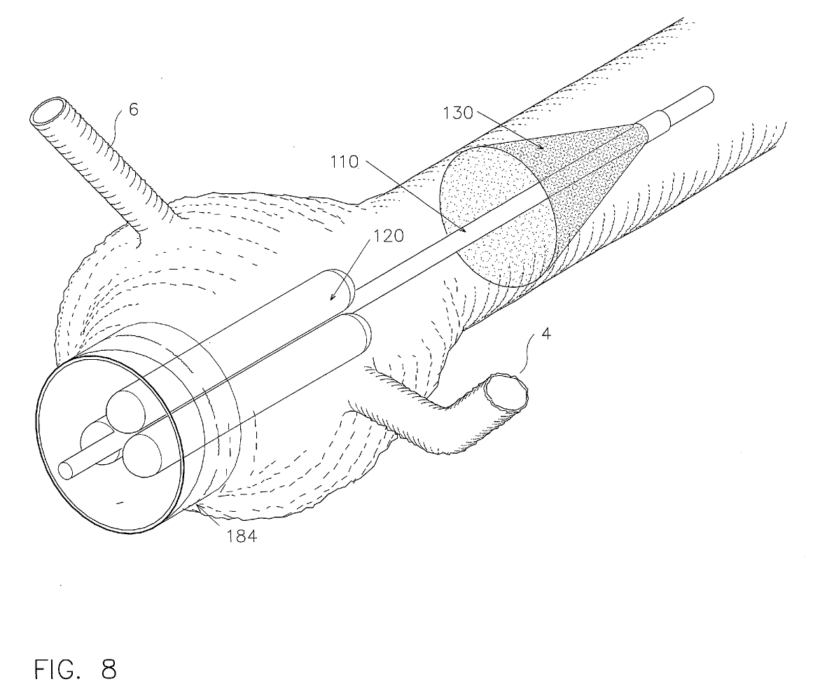

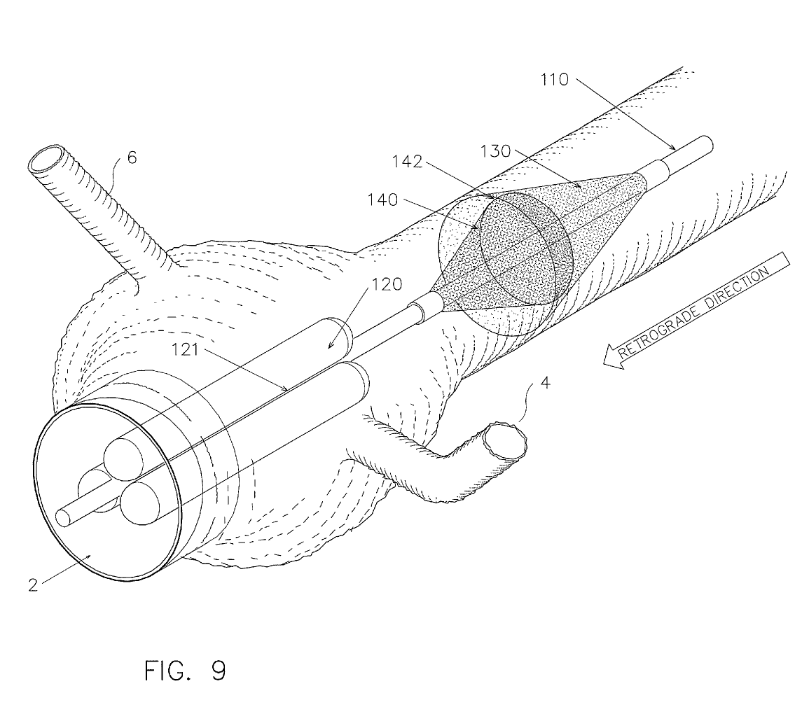

As alluded to above, if desired, a channel 121 can be defined between adjacent inflatable members 120, or adjacent lobes of a single inflatable member to permit perfusion of a patient's blood through the aortic valve during the procedure. At this point in time, debris may be dislodged from filter 130 and sent into the patient's coronary sinus and cerebral arteries, greatly increasing the risk of stroke. However, as depicted in FIG. 8, filter 130 may be deployed at this time, preventing emboli from escaping, thereby protecting the coronary sinus arteries. If filters 130, 140 (or 200) can be moved with respect to inflatable member(s) along elongate body 110, filters can be placed optimally, as can inflatable member(s) 120. As will be appreciated, as depicted in FIG. 9, this second filter 140 can be opened and closed, for example, in response to a change in direction of a patient's bloodflow, a change in the local blood pressure, local pressure gradients in a patient's bloodstream, or a combination of these factors. Preferably, bloodflow progressing in the retrograde direction captures the flared edge 142 of filter 140, encouraging filter 140 to open and thus capture debris that is sent in a retrograde direction out of filter 130, thereby preventing risk of debris entering the coronary sinus and cerebral arteries 4, 6.

The methods and systems of the present invention, as described above and shown in the drawings, provide for a medical device and method for treating the luminal system of a patient with superior properties including, for example, decreased risk of embolism and increased effectiveness for treating cardiac valves of a patient, such as the aortic valve. It will be apparent to those skilled in the art that various modifications and variations can be made in the device and method of the present invention without departing from the spirit or scope of the invention. Thus, it is intended that the present invention include modifications and variations that are within the scope of the appended claims and their equivalents.

What is claimed is:

1. A catheter comprising:
a) an elongate body having a proximal end a distal end, the elongate body defining a longitudinal axis of the catheter;
b) at least one inflatable member disposed on the elongate body proximate the distal end of the elongate body, the interior of the inflatable member being in fluid communication with an inflation lumen in the elongate body;
c) a first filter disposed on the elongate body at a location proximal to the at least one inflatable member, the first filter being adapted and configured to capture emboli in a patient's bloodstream as the bloodstream passes through the first filter along a first direction; and
d) a second filter disposed on the elongate body at a location proximal to the at least one inflatable member and distal to the first filter, the second filter being adapted and configured to capture emboli in a patient's bloodstream as the bloodstream passes through the second filter along a second direction, wherein the second direction is generally opposite to the first direction.

2. The catheter of claim 1, wherein the second filter is adapted and configured to expand and contract along a direction generally transverse to the longitudinal axis of the catheter.

3. The catheter of claim 2, wherein the second filter is adapted and configured to expand and contract in response to a change in direction of a patient's bloodflow.

4. The catheter of claim 2, wherein the second filter is adapted and configured to expand and contract in response to a change in a patient's local blood pressure.

5. The catheter of claim 4, wherein the second filter is adapted and configured to adjust in size in response to local pressure gradients in a patient's bloodstream.

6. The catheter of claim 2, wherein the second filter is adapted and configured to selectively expand and contract.

7. The catheter of claim 1, wherein the second filter is at least partially disposed within the first filter.

8. The catheter of claim 1, wherein the first filter and second filter can be adjusted in size.

9. The catheter of claim 1, wherein the first filter and second filter include radiopaque material.

10. The catheter of claim 1, wherein the first filter and second filter are adapted and configured to be displaced along the longitudinal axis with respect to the at least one inflatable member.

11. The catheter of claim 10, wherein the first filter and second filter are attached to a tubular member that is adapted and configured to receive the elongate body through a lumen defined by the tubular member.

12. The catheter of claim 1, wherein the at least one inflatable member has an undulating exterior surface defining at least one longitudinal channel therein sufficient to permit perfusion in a patient's blood vessel when the at least one inflatable member is expanded.

13. The catheter of claim 1, wherein the catheter includes a plurality of inflatable members that cooperate to define at least one perfusion channel on the exterior of the catheter when the inflatable members are inflated.

14. The catheter of claim 13, wherein the plurality of inflatable members are adapted and configured to close and open the perfusion channel.

15. The catheter of claim 1, wherein the catheter includes a plurality of inflatable members that can be selectively expanded serially or in parallel.

16. The catheter of claim 1, wherein the at least one inflatable member includes polymeric material.

17. The catheter of claim 16, wherein the polymeric material includes polyester.

18. The catheter of claim 16, wherein the polymeric material includes at least one material selected from the group consisting of nylon, polyether block amide, and polyethylene.

19. The catheter of claim 1, wherein the distance between the at least one inflatable member and the second filter is substantially the same as the distance between a patient's aortic valve and the location of the entrance to a patient's coronary sinus.

20. The catheter of claim 1, wherein the distance between the at least one inflatable member and the second filter is adjustable.

21. The catheter of claim 1, wherein the catheter further comprises means for ejecting pressurized liquid proximate the distal end of the catheter.

22. The catheter of claim 21, wherein the means for ejecting liquid includes a plurality of openings in the exterior surface of the catheter in fluid communication with a source of pressurized fluid.

23. The catheter of claim 21, wherein the means for ejecting liquid is adapted and configured to eject liquid in a direction generally transverse to the longitudinal axis.

24. The catheter of claim 21, wherein the means for ejecting liquid includes a plurality of openings on the surface of the inflatable member.

25. The catheter of claim 24, wherein the openings on the surface of the inflatable member are in fluid communication with a source of pressurized fluid that is not in fluid communication with fluid used to inflate the at least one inflatable member.

26. A catheter comprising:
a) an elongate body having a proximal end a distal end, the elongate body defining a longitudinal axis of the catheter;
b) a first filter disposed on the elongate body at a location proximal to the distal end, the first filter being adapted and configured to capture emboli in a patient's bloodstream as the bloodstream passes through the first filter along a first direction; and
c) a second filter disposed on the elongate body at a location proximal to the distal end and distal to the first filter, the second filter being adapted and configured to capture debris in a patient's bloodstream as the bloodstream passes through the second filter along a second direction, wherein the second direction is generally opposite to the first direction.

27. The catheter of claim 26, further comprising at least one inflatable member disposed on the elongate body proximate the distal end of the elongate body, the interior of the inflatable member being in fluid communication with an inflation lumen in the elongate body.

28. The catheter of claim 26, further comprising at least one deliverable prosthesis disposed on the elongate body proximate the distal end of the elongate body, the interior of the inflatable member being in fluid communication with an inflation lumen in the elongate body.

29. The catheter of claim 26, further comprising a removable sheath surrounding at least one of the first filter and second filter.

30. The catheter of claim 29, wherein the sheath surrounds the first filter and second filter.

31. The catheter of claim 30, wherein the second filter is disposed at least partially within the first filter.

32. The catheter of claim 30, wherein the catheter is adapted and configured for a neuro thrombectomy procedure.

33. A method of treating a patient's luminal system, comprising:
a) providing a catheter including:
i) an elongate body having a proximal end a distal end, the elongate body defining a longitudinal axis of the catheter;
ii) at least one inflatable member disposed on the elongate body proximate the distal end of the elongate body;
iii) a first filter disposed on the elongate body at a location proximal to the at least one inflatable member, the first filter being adapted and configured to capture emboli in a patient's bloodstream as the bloodstream passes through the first filter along a first direction; and
iv) a second filter disposed on the elongate body at a location proximal to the at least one inflatable member and distal to the first filter, the second filter being adapted and configured to capture emboli in a patient's bloodstream as the bloodstream passes through the second filter along a second direction, wherein the second direction is generally opposite to the first direction;
b) disposing the distal end of the catheter in a patient's luminal system; and
c) expanding the at least one inflatable member.

34. The method of claim 33, wherein the distal end of the catheter is disposed proximate a valve in the patient's luminal system.

35. The method of claim 34, wherein the valve is the patient's aortic valve.

36. The method of claim 34, further comprising:
a) disposing the first filter at a location downstream from the patient's aortic root to capture emboli; and
b) expanding the second filter to prevent the emboli from being directed into the patient's coronary sinus arteries.

37. The method of claim 36, wherein the second filter is expanded and contracted in response to a change in direction of a patient's bloodflow.

38. The method of claim 36, wherein the second filter is expanded and contracted in response to a change in a patient's local blood pressure.

39. The method of claim 37, wherein the second filter adjusts in size in response to local pressure gradients in a patient's bloodstream.

40. The method of claim 36, wherein the second filter is selectively expanded and contracted.

41. The method of claim 35, further comprising disposing the second filter at least partially within the first filter.

42. The method of claim 33, further comprising displacing the filters along the longitudinal axis with respect to the inflatable member.

43. The catheter of claim 33 further comprising defining a perfusion channel proximate the exterior of the inflatable member.

44. The method of claim 33, further comprising ejecting pressurized liquid proximate the distal end of the catheter to remove debris from the valve area.

45. The method of claim 44, wherein liquid is directed through at least one of the valve leaflets to loosen debris from one or more of the valve leaflets.

46. The method of claim 32, wherein the ejecting step includes directing pressurized liquid through a plurality of openings disposed on the surface of the inflatable member.

47. The catheter of claim 11, wherein the first filter and second filter can be moved closer to or further away from each other along the axis of the catheter.
